(19) United States
(12) Patent Application Publication
(10) Pub. No.: US 2010/0030057 A1
(43) Pub. Date: Feb. 4, 2010

(54) IMAGING CATHETER

(76) Inventors: Oren Gavriely, Haifa (IL); Ofer Fridman, Lotem (IL)

Correspondence Address: YORAM TSIVION, P.O. BOX 1307, PARDES HANNA, 37111, IL

(21) Appl. No.: 11/568,046

(22) PCT Filed: Apr. 19, 2005
(86) PCT No.: PCT/IL05/00410

§ 371 Date: Oct. 18, 2006

Related U.S. Application Data
(60) Provisional application No. 60/564,246, filed on Apr. 19, 2004.

(51) Int. Cl. A61M 25/00; A61M 25/09; A61B 6/00

(52) U.S. Cl. 600/407; 604/523; 604/528

(57) ABSTRACT

A catheter for conveying flowables in or out of the body, be they gases or liquids. The catheter has a triangular lumen and optional auxiliary structures. One optional element is guide engaging for employing a guide for placing the catheter. Another element is a secondary lumen disposed within the wall of the tube. The camera and lighting devices at the tip of the catheter are mounted on a partially flexible PCB and packaged in the tube permitting all elements to point substantially forwards.

### TECHNICAL FIELD OF THE INVENTION

The present invention relates generally to medical devices and more specifically to imaging catheters.

### BACKGROUND OF THE INVENTION

Insertion of tubes into patient's body organs, cavities and tracts is a common procedure in emergency and critical care medicine. An endotracheal tube may be inserted into the trachea of a patient who is in acute respiratory failure or is undergoing general anesthesia. The endotracheal tube must be placed quickly and accurately and positioned with its tip in the mid portion of the patient's trachea to prevent accidental slipping and to provide proper seal and ventilation of both lungs. Similarly, a naso-gastric tube is commonly inserted through the nose or mouth into the stomach of patients who need artificial feeding or evacuation of the content of the stomach. Another tube that is frequently inserted into a body cavity during emergency treatment is the urinary catheter. This catheter is threaded through the urethra into the urinary bladder. The correct placement of these tubes and catheters throughout their use is critically important. Examples of other catheters are peritoneal dialysis/lavage catheter, chest tube, and rectal tube.

Many patients who are critically ill or undergoing general anesthesia require artificial ventilation. For over 40 years the most common method of providing artificial ventilation has been by pumping compressed air into the patient's lungs through an endotracheal tube. This tube is inserted through the patient's mouth or nose and passed between the vocal cords into the trachea. Alternatively, a tube may be inserted into the trachea through a tracheotomy surgical incision. For oral intubation the operator usually uses a laryngoscope, which consists of a handle and a blade. The operator inserts the blade into the patient's mouth and advances it until its tip lies in the pharynx beyond the root of the tongue. The handle is then used to manipulate the blade and push the tongue out of the way until the epiglottis and the vocal folds can be seen. The tip of the endotracheal tube can then be aimed and pushed between the vocal folds into the trachea. This method of insertion is used in the majority of intubations, but requires skill, training and experience and is only performed by specialized physicians and licensed paramedics.

An alternative method that is often used when difficult intubation is anticipated is over a fiber optic bronchoscope. First the bronchoscope is connected to a light source to provide the needed illumination of the field facing its tip. The shaft of the bronchoscope is then inserted through the endotracheal tube and moved in as far as possible. The tip of bronchoscope is then inserted into the patient's airway and advanced under visualization through the bronchoscope's eyepiece or a video display in between the vocal folds into the trachea. The endotracheal tube can now be pushed down the bronchoscope shaft and moved between the vocal folds into the trachea. The endotracheal tube can now be secured and the bronchoscope removed to free up the lumen of the endotracheal tube. While the bronchoscopic method is safer than with the laryngoscope, the equipment needed is expensive, delicate and more cumbersome and is seldom found in the field or on emergency medical vehicles.

Securing the endotracheal tube and preventing its inadvertent movement during use is critical to the prevention of dire accidents. Inflating a cuff that surrounds the tube near its tip occludes the space between the outer wall of the tube and the inner wall of the trachea to provide an airtight seal. The cuff is connected to the external end of the endotracheal tube through a thin channel in the tube's wall. The channel is connected to a one-way valve through which air can be injected to inflate the cuff to the desired pressure and volume. The cuff is also helpful in securing the tube in place, but additional fasteners are usually applied around the head to prevent the tube from slipping in or dislodging.

Once the tube has been inserted, it is necessary to verify its correct position. Accidental insertion of the tube into the esophagus or placing it too deep inside the airways, so that its tip is lodged in one of the main stem bronchi instead of in the trachea may lead to catastrophic consequences and asphyxiation. Many methods are available to verify the endotracheal tube placement. Auscultation of both sides of the chest is usually done to verify symmetric air entry into both lungs. A chest x-ray is another well-tested method of verifying the tube placement. The x-ray picture reveals the relationships between the endotracheal tube tip and the tracheal first bifurcation (carina). X-ray pictures may be and should be taken whenever an endotracheal tube is placed or repositioned. Additionally, the tube placement may be verified through a fiber optic bronchoscope, by a suction bulb, or through sending and receiving an acoustic signal. These methods are used to verify the initial placement of the endotracheal tube. There are no currently available means for continuous monitoring of the actual placement of the tube.

The advantages of fiber optic visualization were combined with the simple design of the laryngoscope as disclosed by several patents and scientific papers. Additionally, the use of visualization stylets which include means for seeing the airways during the insertion of an endotracheal tube have been described. However, there are no known methods for incorporating the visualization means permanently into the anterior face of the endotracheal tube and to insert it in with out the laryngoscope.

Tubes that are inserted into a body cavity are usually made of soft materials that have to be flexible and deformable to conform to the contours of the tract through which they are inserted and to reduce to a minimum the pressure on and friction with the contacting tissues. However, soft-wall tubes are prone to bending, collapse and kink formation during their insertion and thereafter.

To prevent collapse, some tubes are made of more rigid materials, or with thicker walls with the inherent disadvantage of causing potential pressure injury to the tissue. Other tubes, which are currently made from soft materials, such as silicone, are equipped with internal scaffold made from harder materials. For example, there are endotracheal tubes that have an embeded spiral made of flexible still. The spiral prevents collapse while maintaining axial flexibility of the tube. The internal spiral substantially increases the complexity of the tube manufacturing and its cost.

The difficulty of insertion of a soft tube into the body is usually overcome by an internal semi rigid introducer, or stilet that is passed through the tube. This kind of internal introducers obliterate the lumen of the tube and prevent its use for conveying fluids during the insertion process. Another method for facilitating the insertion process is by use of accessories that dilate the tissues and create a space through which the tube can be passed with minimal resistance. Such accessories include the laryngoscope and the Magil forceps.

### BRIEF DESCRIPTION OF THE DRAWINGS

FIG. 1 is a schematic isometric description of a tube of the invention showing a slanted face of proximal ending;

FIG. 2A is a schematic isometric description of a tip of a tube of the invention indicating a guide engagement structure element;

FIG. 2B is a schematic isometric description of a tip of a tube of the invention indicating a guide engagement structure element;

FIG. 2C is a schematic isometric description of a tip of a tube of the invention indicating a guide engagement structure element;

FIG. 2D is a schematic isometric description of a tip of a tube of the invention indicating a guide engagement structure element;

FIG. 2E is a schematic isometric description of a tube of the invention, and a guide having gripping wings;

FIG. 2F is a schematic isometric description of a tube of the invention, and a guide bearing a handle;

FIG. 3 is a schematic cross sectional view in a catheter of the invention employing an inflating sleeve protected by a protective sleeve;

FIG. 4A is an isometric schematic description of a light source and imaging sensor assembly employing a flexible PCB;

FIG. 4B is an isometric schematic description of a light source and imaging sensor assembly employing a flexible PCB packaged inside a catheter.

### DETAILED DESCRIPTION OF THE INVENTION

### General Tube Structure

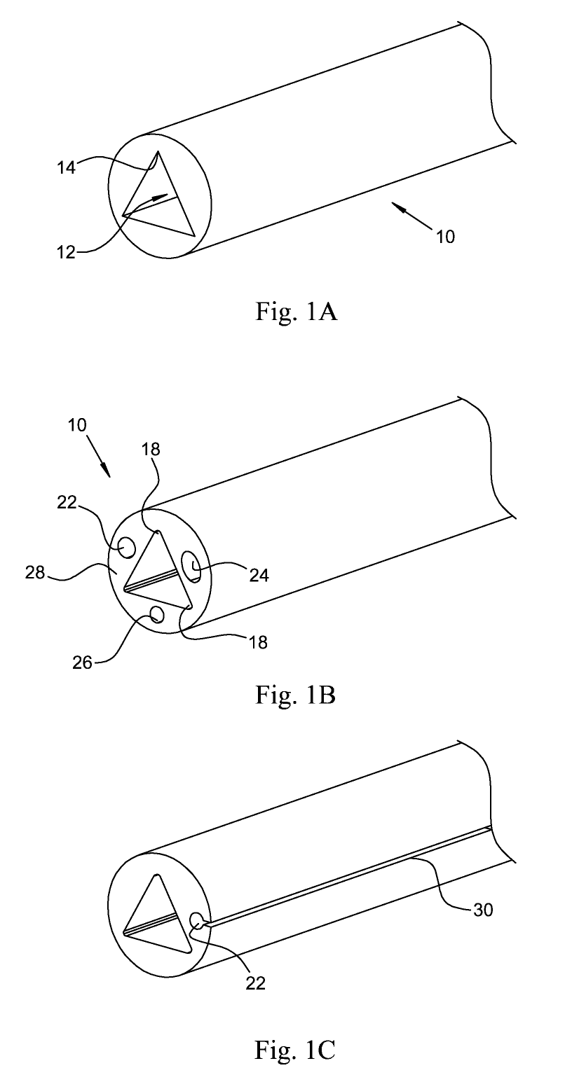

To overcome the problems outlined above we disclose a tube for insertion into a cavity in the human or animal body made of flexible and soft materials such as silicone, polyethylene, latex, PVC or similar elastomers. The tube of the invention has a round or triangular lumen and a regular round external circumference of the tube r conveys flowables (liquids or gases) into or out of a cavity of the body or from one site to another site inside or out side the body. The proximal ending (the end inserted into the body of the patient) is either vertical or slanted. In FIG. 1 a proximal ending 10 of a tube used in an embodiment of the invention is shown, in which the face 28 of which is slanted.

### The Guide

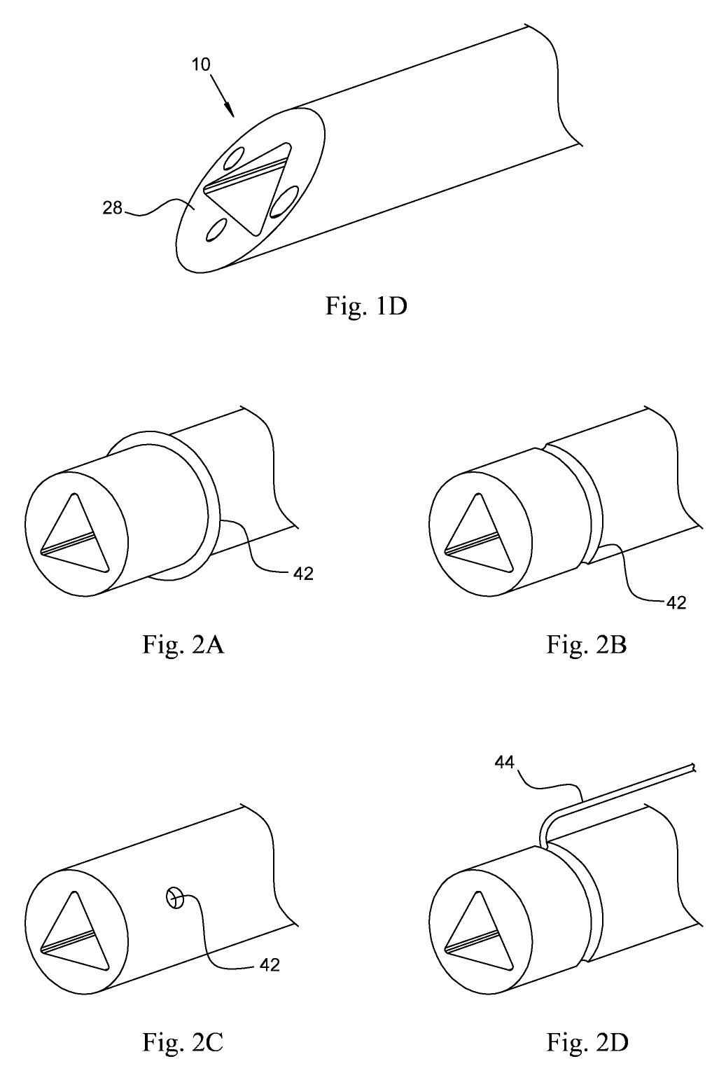

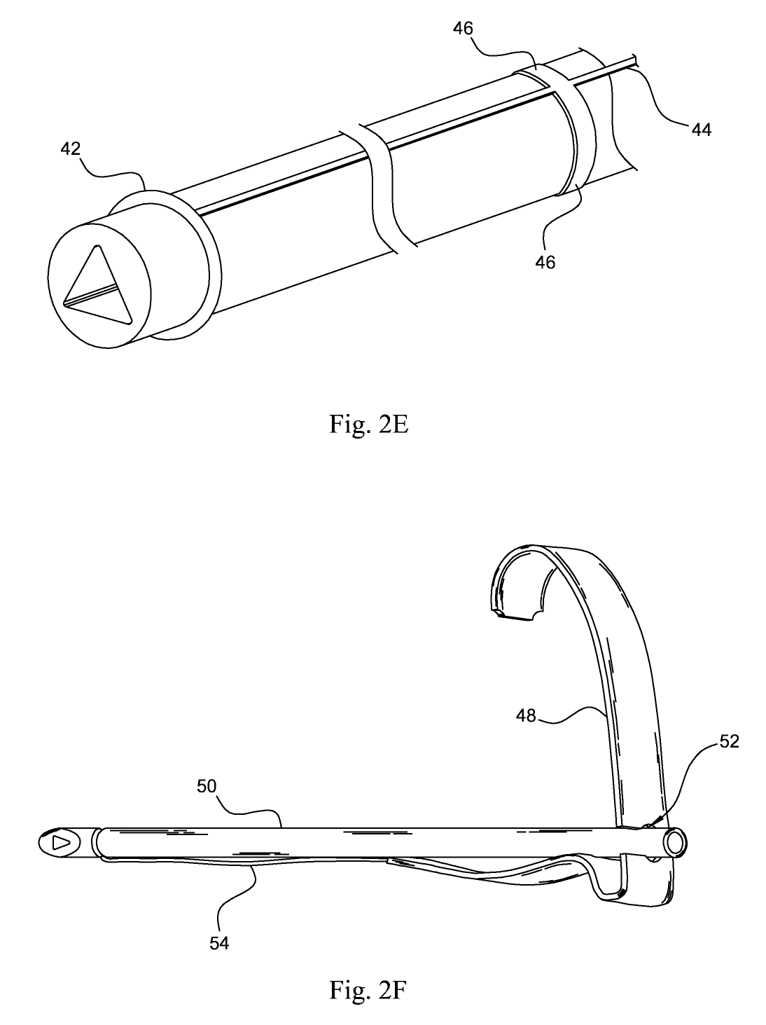

One aspect of the invention is a means for guiding a flexible tube into a cavity in the body of a person or an animal. In accordance with one aspect of the invention, a guide assists in the placement of the flexible tube. The guide is a rigid or semi rigid elongated tool, typically metal or plastic, that is either inserted in the lumen, or secondary lumen, or in an external slit or in an internal slit or is entirely is external, attached internally to the internal surface of the tube or superficially to the external surface of the tube. An externally disposed guide may be attached to the tube at one or more sites along the shaft of the tube. A guide may also be, generally stated, disposed part of the way inside and part of the way in the lumen or slit of the tube. To accomplish guidance, the guide of the invention is engaged with the tip of the tube such that once placed, it can be readily removed. As can be seen in FIGS. 2A-F, a guide engagement structural element for accomplishing the guidance is formed in connection with the tube. In FIG. 2A the engagement structure element 42 is a peripheral ring surrounding the tube near the tip. In FIG. 2B the engagement structure element 42 is a peripheral groove surrounding the tube near its tip. In FIG. 2C the engagement element 42 is a pit in the tube. Yet another engagement element is a fold. In FIG. 2D the engagement element is a groove, in which the tip of the guide is inserted, allowing a forward push by the guide 44. In FIG. 2E the engagement element 42 is pushable by guide 44. The guide wire bearing two tube—gripping wings 46, and is situated at such a distance from the engagement point as to stabilize the guide along the shaft of the tube. In some embodiments, the guide is attached to a handle which facilitates easy twisting for example. Some handles may be bent forming a circular structure for gripping the catheter to assist guiding and or releasing. In FIG. 2F a handle 48 is shown which grasps the tube 50 snugly in a notch 52. To release the guide 54, the handle is twisted sideways and the guide pulled back.

The engagement of the guide with the tube is temporary, so that after the completion of the insertion process, the guide may be disconnected from tube and removed. The disconnection is accomplished by drawing the guide back, or by twisting and drawing back. With guides having attachment wings, the guide is drawn back by pulling along the shaft of the tube until the attaching wings are freed from the hold of the tube. If the wings are not too tight, the tube can be freed by pulling sideways. In such cases as the guide is inserted through a secondary lumen, or in a slit alongside the tube, it is usually inserted only part of the way through the tube. In such cases there is an insertion hole in the wall of the tube connecting the outside with a lumen or a slit.

### Sleeve Protection

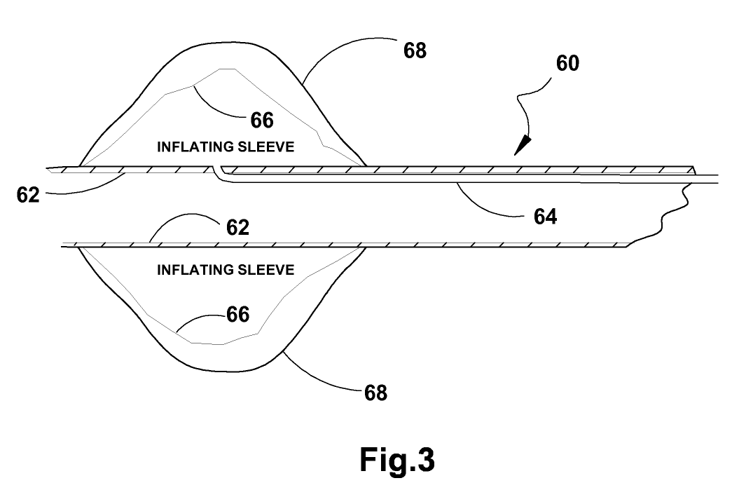

The use of an inflating cuff or sleeve for stabilizing catheters is well known, for example such a device is disclosed in international patent application WO 2004/043527. The cuff/sleeve is used to anchor a catheter and/or block a passageway by inserting in tubular structures, such that by inflating the cuff/sleeve, a firm internal hold is accomplished by the catheter's cuff/sleeve pushing radially against the structure's walls. (In accordance with an embodiment of the present invention the cuff/sleeve is protected by an external sleeve as described in FIG. 3 to which reference is now made. A longitudinal section in tube 60 shows the tube wall 62 (hatched), a sleeve inflating tube 64, which in other embodiments is embedded within the tube wall, and the sleeve 66. A protective sleeve 68 covers the inflating sleeve. The protective sleeve imparts mechanical protection to the sleeve inflating tube. When a guide is used with a sleeved tube, there are two main possible arrangements to accommodate the guide. According to one arrangement, an elongated bag is inserted alongside the tube such that it is fully covered by the cuff except for the opening through which the guide is inserted. In another arrangement the guide is inserted through hole in the side of the tube that connects to a secondary lumen. The sleeve in such a case, may cover the entrance hole after being inflated, which block the entrance and prevents contamination in the secondary lumen.

### Lighting and Imaging Sensor Assembly and Packaging

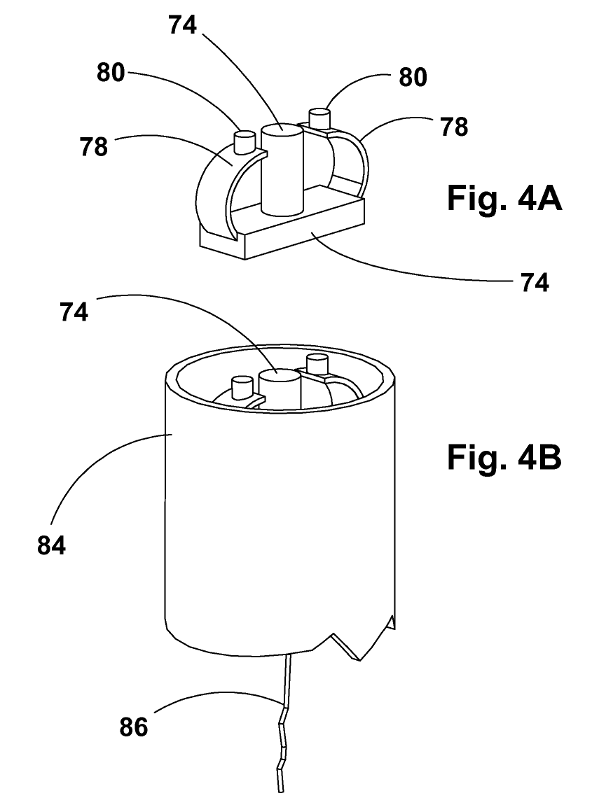

The catheter of the invention is particularly beneficial with a lighting unit that is useful for reasons such as maneuvering the catheter and monitoring internal parts of the body. An imaging sensor is installed at the tip of the catheter of the invention as described with reference to FIGS. 4A-B to which reference is now made. FIG. 4A shows that imaging sensor and lens 74 is assembled on a miniature PCB (printed circuit board) 76. On the flexible PCB flaps 78 are mounted light sources, typically LEDs (light emitting diodes) 80. This particular assembly of imaging sensor and lighting elements facilitates the disposal of the light sources in substantially the same level as the imaging sensor lens. In other examples, different levels, below or above, of the lighting elements with respect to the imaging sensor can be implemented. In FIG. 4B the imaging sensor assembly is shown packaged within the catheter tube 84, such that the imaging sensor 74 and the entire assembly is kept in proper angles, typically directing the lighting elements forwards. The feed wire 86 runs along the lumen of tube 84. In a preferred embodiment of the invention, the wire feeding the imaging sensor is different than the wire feeding the light source. The separate feeding facilitates implementing different energizing regimes for the lighting elements than for the imaging sensor. For example, to avoid excessive heating, the light might be cyclically interrupted, or dimmed, or some of the lighting elements may be turned off, or different light spectra may be used, without concomitant changes being made to the imaging sensor.

### Benefits of the Triangular Internal Circumference

An internally triangular tube having the same mass per unit length as a an internally circular tube has a higher bending stiffness, resistance to collapse and prevention of kinking An additional advantage of the internally triangular lumen tube is that it can accommodate for secondary lumens of substantial width. The essentially triangular lumen of tubes of the invention is applicable to all flexible wall tubes used fro inserting in the body, including, but not limited to endotracheal tubes, naso-gastric tube, chest tube, peritoneal tube, urinary catheter, rectal tube. This form of lumen is also applicable to tubes that are implanted in the body in order to translocate fluids from one cavity to another such as a tube used to shunt cerebrospinal fluid from the brain ventricles to the chest or abdominal cavity.

1. A catheter having at least one guide engagement structure element is disposed on its surface.

2. A guide for placing a catheter's tip by provisionally engaging with a structure of said catheter, wherein said guide is substantially elongated and rigid.

3. A guide for placing a catheter's tip as in claim 6 and wherein said guide bears tube gripping wings.

4. A guide for placing a catheter's tip as in claim 6 and wherein said guide is attached to a handle.

5. A guide for placing a catheter's tip as in claim 8 and wherein said handle employs a means for grasping said guide.

6. A lighting and imaging sensor assembly, whereby at least one item contained in the list consisting of imaging sensors and lighting devices is assembled on a miniature PCB and at least one item contained in the list consisting of imaging sensors and lighting devices, is assembled on a flexible PCB flap of said PCB.

7. A lighting and imaging sensor assembly as in claim 10 wherein said PCB and said PCB flap are packaged in a catheter.

8. A lighting and imaging sensor assembly wherein different circuits energize said at least one lighting element and said at least one imaging sensor respectively.

9. A catheter for conveying flowables to sites from sites selected from the group consisting of inside the body to the outside of the body, a first site to a second site both within the body, outside of the body to a site within the body, wherein said catheter employs an inflatable sleeve, and wherein said catheter bears a protective sleeve externally disposed over said inflatable sleeve.
